(19) United States
(12) Patent Application Publication
(10) Pub. No.: US 2025/0022561 A1
(43) Pub. Date: Jan. 16, 2025

(54) SYSTEMS AND METHODS FOR IMPROVING INTERACTIONS OF INSURANCE PROVIDERS, PRESCRIBING HEALTHCARE PROFESSIONALS, AND MEMBERS ASSOCIATED WITH A HIGH VOLUME PHARMACY

(71) Applicant: Express Scripts Strategic Development, Inc., St. Louis, MO (US)

(72) Inventors: Peter A. Rosomoff, Wildwood, MO (US); Mahsa Shateri, Los Angeles, CA (US)

(21) Appl. No.: 18/220,936

(22) Filed: Jul. 12, 2023

(51) Int. Cl. G16H 20/10; G16H 80/00

(52) U.S. Cl. CPC G16H 20/10; G16H 80/00

(57) ABSTRACT

A method includes receiving prescription data associated with a plurality of prescriptions written by a prescribing healthcare professional and determining a prescriber score for the prescribing healthcare professional based on the prescription data. The method also includes determining a prescription product score for each prescription product, retrieving member data, and determining a member score for each respective member. The method also includes determining a prescriber propensity score for the prescribing healthcare professional, and, in response to a determination that the prescriber propensity score is greater than a first threshold, identifying respective members having a member score that is greater than a second threshold, and generating, for display, an output indicating at least the prescriber propensity score and a list of identified respective members.

### CROSS-REFERENCE TO RELATED APPLICATIONS

This application is related to U.S. patent application Ser. No. ______, filed ______, 2023, and entitled “SYSTEMS AND METHODS FOR IMPROVING INTERACTIONS OF INSURANCE PROVIDERS, PRESCRIBING HEALTHCARE PROFESSIONALS, AND MEMBERS ASSOCIATED WITH A HIGH VOLUME PHARMACY” (Attorney Docket No. ESRX-383US2), and to U.S. patent application Ser. No. ______, filed ______, 2023, and entitled “SYSTEMS AND METHODS FOR IMPROVING INTERACTIONS OF INSURANCE PROVIDERS, PRESCRIBING HEALTHCARE PROFESSIONALS, AND MEMBERS ASSOCIATED WITH A HIGH VOLUME PHARMACY” (Attorney Docket No. ESRX-383US3), which are incorporated by reference herein.

### TECHNICAL FIELD

This disclosure relates to high volume pharmacies, and in particular to systems and methods for improving interactions of insurance providers, prescribing healthcare professionals, and members associated with a high volume pharmacy.

### BACKGROUND

Medications, such as prescription medications, over-the-counter medications, vitamins, supplements, and the like, are typically provided by a medication provider, such as a large volume pharmacy and the like. A healthcare processional, such as a physician, may write a various prescriptions for a patient to provide therapeutic treatment, using a medication associated with a prescription, for corresponding ailments of the patient. A prescription may include non-generic and/or generic versions of a medication, which may include a therapeutic alternative to the non-generic version of the medication.

Typically, the patient, using insurance, may have a lower co-pay or an overall lower out of pocket expense for a generic version of a medication relative to the corresponding non-generic version of the medication. As such, healthcare professionals may identify a generic version of a medication as a therapeutic alternative to the non-generic version of the medication in order to reduce the healthcare expenses of the patient. Additionally, members may benefit from home delivery of various medications and/or other prescription accessories.

### SUMMARY

This disclosure relates generally to high volume pharmacy interactions.

An aspect of the disclosed embodiments includes a system for determining a propensity of a prescribing healthcare professional to write a prescription for home delivery. The system includes: a processor; and a memory including instructions that, when executed by the processor, cause the processor to: receive prescription data associated with a plurality of prescriptions written by a prescribing healthcare professional; determine a prescriber score for the prescribing healthcare professional based on the prescription data; determine a prescription product score for each prescription product in each respective prescription of the plurality of prescriptions associated with the prescription data; retrieve, from a member database, member data associated with respective members of a plurality of members represented in the member database, wherein the respective members have a treatment association with the prescribing healthcare professional; determine a member score for each respective member of the plurality of members based on at least one home delivery factor; based on the prescriber score, each prescription product score, and each member score, determine, using an artificial intelligence engine configured to use at least one machine learning model, a prescriber propensity score for the prescribing healthcare professional; and, in response to a determination that the prescriber propensity score is greater than a first threshold: identify respective members having a member score that is greater than a second threshold; and generate, for display, an output indicating at least the prescriber propensity score and a list of identified respective members.

Another aspect of the disclosed embodiments includes a method for determining a propensity of a prescribing healthcare professional to write a prescription for home delivery. The method includes: receiving prescription data associated with a plurality of prescriptions written by a prescribing healthcare professional; determining a prescriber score for the prescribing healthcare professional based on the prescription data; determining a prescription product score for each prescription product in each respective prescription of the plurality of prescriptions associated with the prescription data; retrieving, from a member database, member data associated with respective members of a plurality of members represented in the member database, wherein the respective members have a treatment association with the prescribing healthcare professional; determining a member score for each respective member of the plurality of members based on at least one home delivery factor; based on the prescriber score, each prescription product score, and each member score, determining, using an artificial intelligence engine configured to use at least one machine learning model, a prescriber propensity score for the prescribing healthcare professional; and, in response to a determination that the prescriber propensity score is greater than a first threshold: identifying respective members having a member score that is greater than a second threshold; and generating, for display, an output indicating at least the prescriber propensity score and a list of identified respective members.

Another aspect of the disclosed embodiments includes an apparatus for determining a propensity of a prescribing healthcare professional to write a prescription for home delivery. The apparatus includes: one or more processors; and at least one memory including instructions that, when executed by the one or more processors, cause the one or more processors, collectively or respectively, to: determine a prescriber score for a prescribing healthcare professional based on prescription data associated with a plurality of prescriptions written by the prescribing healthcare professional; determine a prescription product score for each prescription product in each respective prescription of the plurality of prescriptions associated with the prescription data; retrieve, from a member database, member data associated with respective members of a plurality of members represented in the member database, wherein the respective members have a treatment association with the prescribing healthcare professional; determine a member score for each respective member of the plurality of members based on at least one home delivery factor; based on the prescriber score, each prescription product score, and each member score, determine, using an artificial intelligence engine configured to use at least one machine learning model, a prescriber propensity score for the prescribing healthcare professional; and, in response to a determination that the prescriber propensity score is greater than a first threshold: identify respective members having a member score that is greater than a second threshold; and generate, for display, an output indicating at least the prescriber propensity score and a list of identified respective members.

Another aspect of the disclosed embodiments includes a system for identifying combinations of prescribing healthcare professionals, members, and products for targeted communication. The system includes: a processor; and a memory including instructions that, when executed by the processor, cause the processor to: receive a plurality of prescriber scores for respective prescribing healthcare professionals; receive a plurality of prescription product scores for respective prescription products; receive a plurality of member scores for receptive members, each respective member being associated with at least one of at least one prescription product associated with a prescription product score of the plurality of prescription product scores and at least one prescribing healthcare professional associated with a prescriber score of the plurality of prescriber scores; receive targeted communication input indicating a prescriber score weight, a prescription product score weight, and a member score weight; identify at least one combination of a prescribing healthcare professional, a prescription product, and a member using an artificial intelligence engine configured to use at least one machine learning model configured to use the prescriber score, the prescriber score weight, the prescription production score, the prescription product score weight, the member score, and the member score weight; and generate, for display, an output indicating at least the at least one combination of a prescribing healthcare professional, a prescription product, and a member.

Another aspect of the disclosed embodiments includes a method for identifying combinations of prescribing healthcare professionals, members, and products for targeted communication. The method includes: receiving a plurality of prescriber scores for respective prescribing healthcare professionals; receiving a plurality of prescription product scores for respective prescription products; receiving a plurality of member scores for receptive members, each respective member being associated with at least one of at least one prescription product associated with a prescription product score of the plurality of prescription product scores and at least one prescribing healthcare professional associated with a prescriber score of the plurality of prescriber scores; receiving targeted communication input indicating a prescriber score weight, a prescription product score weight, and a member score weight; identifying at least one combination of a prescribing healthcare professional, a prescription product, and a member using an artificial intelligence engine configured to use at least one machine learning model configured to use the prescriber score, the prescriber score weight, the prescription production score, the prescription product score weight, the member score, and the member score weight; and generating, for display, an output indicating at least the at least one combination of a prescribing healthcare professional, a prescription product, and a member.

Another aspect of the disclosed embodiments includes an apparatus for identifying combinations of prescribing healthcare professionals, members, and products for targeted communication. The apparatus includes: one or more processors; and at least one memory including instructions that, when executed by the one or more processors, cause the one or more processors, collectively or respectively, to: determine a plurality of prescriber scores for respective prescribing healthcare professionals; determine a plurality of prescription product scores for respective prescription products; determine a plurality of member scores for receptive members, each respective member being associated with at least one of at least one prescription product associated with a prescription product score of the plurality of prescription product scores and at least one prescribing healthcare professional associated with a prescriber score of the plurality of prescriber scores; receive targeted communication input indicating a prescriber score weight, a prescription product score weight, and a member score weight; identify at least one combination of a prescribing healthcare professional, a prescription product, and a member using an artificial intelligence engine configured to use at least one machine learning model configured to use the prescriber score, the prescriber score weight, the prescription production score, the prescription product score weight, the member score, and the member score weight; and generate, for display, an output indicating at least the at least one combination of a prescribing healthcare professional, a prescription product, and a member.

Another aspect of the disclosed embodiments includes a system for insurance member communication. The system includes: a processor; and a memory including instructions that, when executed by the processor, cause the processor to: receive a plurality of member communication campaign input files that define a set of pharmacy product exclusion rules and a set of pharmacy product inclusion rules, wherein the plurality of member communication campaign input files are associated with a member communication campaign; generate one or more campaign tables in a campaign table database based the set of pharmacy product exclusion rules and the set of pharmacy product inclusion rules; retrieve, from an insurance claim database and based on the plurality of member communication campaign input files, insurance claim data; populate records of the one or more campaign tables with the insurance claim data; suppress data in the one or more campaign tables based on a set of suppression rules; and for at least one record of the one or more campaign tables: identify a pharmacy used by an associated member to fill one or more prescriptions; identify one or more alternative pharmacies within a predetermined distance from the identified pharmacy; identify at least one product of the one or more prescriptions; identify one or more alternative products associated with the at least one product of the one or more prescriptions; generate a communication data object that includes (i) at least one alternative pharmacy of the one or more alternative pharmacies that meet at least one alternative pharmacy criteria and (ii) at least one alternative product of the one or more alternative products that meet at least one alternative product criteria; and communicate the communication data object to the associated member.

Another aspect of the disclosed embodiments includes a method for insurance member communication. The method includes: receiving a plurality of member communication campaign input files that define a set of pharmacy product exclusion rules and a set of pharmacy product inclusion rules, wherein the plurality of member communication campaign input files are associated with a member communication campaign; generating one or more campaign tables in a campaign table database based the set of pharmacy product exclusion rules and the set of pharmacy product inclusion rules; retrieving, from an insurance claim database and based on the plurality of member communication campaign input files, insurance claim data; populating records of the one or more campaign tables with the insurance claim data; suppressing data in the one or more campaign tables based on a set of suppression rules; and for at least one record of the one or more campaign tables: identifying a pharmacy used by an associated member to fill one or more prescriptions; identifying one or more alternative pharmacies within a predetermined distance from the identified pharmacy; identifying at least one product of the one or more prescriptions; identifying one or more alternative products associated with the at least one product of the one or more prescriptions; generating a communication data object that includes (i) at least one alternative pharmacy of the one or more alternative pharmacies that meet at least one alternative pharmacy criteria and (ii) at least one alternative product of the one or more alternative products that meet at least one alternative product criteria; and communicating the communication data object to the associated member.

Another aspect of the disclosed embodiments includes an apparatus for insurance member communication. The apparatus includes: one or more processors; and at least one memory including instructions that, when executed by the one or more processors, cause the one or more processors, collectively or respectively, to: generate one or more campaign tables in a campaign table database based a set of pharmacy product exclusion rules and a set of pharmacy product inclusion rules; retrieve, from an insurance claim database, insurance claim data; populate records of the one or more campaign tables with the insurance claim data; suppress data in the one or more campaign tables based on a set of suppression rules by removing the suppressed data from the one or more campaign tables and loading the suppressed data in a drop table of the campaign tables database; and for at least one record of the one or more campaign tables: identify a pharmacy used by an associated member to fill one or more prescriptions; identify one or more alternative pharmacies within a predetermined distance from the identified pharmacy; identify at least one product of the one or more prescriptions; identify one or more alternative products associated with the at least one product of the one or more prescriptions; generate a communication data object that includes (i) at least one alternative pharmacy of the one or more alternative pharmacies that meet at least one alternative pharmacy criteria and (ii) at least one alternative product of the one or more alternative products that meet at least one alternative product criteria; and communicate the communication data object to the associated member.

These and other aspects of the present disclosure are disclosed in the following detailed description of the embodiments, the appended claims, and the accompanying figures.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A generally illustrates a functional block diagram of a system including a high-volume pharmacy according to the principles of the present disclosure.

FIG. 1B generally illustrates a computing device according to the principles of the present disclosure.

FIG. 2 generally illustrates a functional block diagram of a pharmacy fulfillment device, which may be deployed within the system of FIG. 1A.

FIG. 3 generally illustrates a functional block diagram of an order processing device, which may be deployed within the system of FIG. 1A.

FIG. 4 is a flow diagram generally illustrating a prescriber home delivery propensity determination method according to the principles of the present disclosure.

FIG. 5 is a flow diagram generally illustrating a member targeting method according to the principles of the present disclosure.

FIG. 6 is a flow diagram generally illustrating a member communication generation method according to the principles of the present disclosure.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

As described, medications, such as prescription medications, over-the-counter medications, vitamins, supplements, and the like, are typically provided by a medication provider, such as a large volume pharmacy and the like. A healthcare processional, such as a physician, may write a various prescriptions for a patient to provide therapeutic treatment, using a medication associated with a prescription, for corresponding ailments of the patient. A prescription may include non-generic and/or generic versions of a medication, which may include a therapeutic alternative to the non-generic version of the medication.

Typically, the patient, using insurance, may have a lower co-pay or an overall lower out of pocket expense for a generic version of a medication relative to the corresponding non-generic version of the medication. As such, healthcare professionals may identify a generic version of a medication as a therapeutic alternative to the non-generic version of the medication in order to reduce the healthcare expenses of the patient. Additionally, members may benefit from home delivery of various medications and/or other prescription accessories.

Increasingly, high volume pharmacies and/or insurance providers are reaching out to members or customers to encourage use of various services or products, to improve customer satisfaction, to encourage the use of various in network services, and the like. This may include using various systems to identify members or customers for targeted communication. For example, such systems typically receive a list of patients that have not been targeted in the past period (e.g., such as 6 months or other suitable period) and had retail activities on a prescription that has a profit that is equal to or greater than a predefined conversation profit margin. The system may use a dialer to make multiple attempts to reach a patient. Reaching the patient may be difficult with such a dial because the patient may not be available or the patient may ignore the call.

In cases where the system is able to reach the patient, the system may present the patient with an opportunity to improve the experience of the patient. For example, the system may offer to change one or more prescriptions of the pantie to home delivery. However, the patient may either accept or reject such an opportunity. Once the patient accepts the opportunity, the system makes multiple attempts to reach an associated physician. However, similar to patient communication, they are instances that prescriber will not accept the conversion due to various reasons. Historical data indicates that for 35-40% of opportunities in which the patient has accepted the conversion, the new script from the physicians has never received.

Typically, once the patient accepts the opportunity, the prescriber is given up to a threshold number of days (e.g., 21 days or other suitable number of days) to decide if the prescription request can be converted to mail order for home (e.g., or other location) delivery. From the patient perspective, who is excited to not only save money but also save the trip to the pharmacy, the wait-time for the threshold number of days can be frustrating. Moreover, in cases where the physicians does not respond to the prescription conversion request, the patient may be very dissatisfied from the missed opportunity, especially if the patient has waited the threshold number of days. Targeting a population with higher chance of success rate is important. Some prescribing healthcare professionals do not need any interventions for the prescription approval, while others need significant intervention and phone calls before the prescribing healthcare professional responses.

Accordingly, systems and methods, such as those described herein, configured to improve interactions between insurance providers, prescribing healthcare professionals, and members (e.g., which may be referred to herein as patients, users, and/or the like), may be desirable. In some embodiments, the systems and methods described herein may be configured to select a target list based on the prescription conversion profit margin. The systems and methods described herein may be configured to, generate the target list based on the highest margin received on the prescription at the time of conversation (e.g., retail store to mail delivery).

In some embodiments, the systems and methods described herein may be configured to combine the physician-drugs-and patient with the current supply chain target list to drive a higher expected value of margin and probability of success. Prescribing healthcare professional responses may be categorized based on a respective response rate and turnaround times. Some prescribing healthcare professionals commonly respond to most retail to mail requests within a short amount of time and some prescribing healthcare professionals ignore the retail to mail requests or rarely respond after many days. The systems and methods described herein may be configured to monitor and track behavior of the prescribing healthcare professionals over time. If the pattern of behavior remains the same over period (e.g., 3 years or other period), the systems and methods described herein may be configured to adjust or monitor parameters associated with a respective prescribing healthcare professional. Alternatively, if the patterns change in recent activities, depending on the consistency in declined and/or improved trend and frequency of recent activities, the systems and methods described herein may be configured to assign different scoring loads to the most recent data.

The systems and methods described herein may be configured to weigh, seperatly, historical and recent percent acceptance rates, turnaround times, familiarity of the prescribing healthcare professional with the systems and methods described herein, therapies, and number of intervention. The systems and methods described herein may be configured to dynamically balance weights based on a factor of importance and tenure of the patient and/or doctors.

In some embodiments, the systems and methods described herein may be configured to, analyze therapy level information for various drugs and determine which drugs are safer to be target (e.g., for conversion from retail to mail delivery), which drugs are most preferred for the prescribing healthcare professionals based on the pattern of behavior of particular prescribing healthcare professionals, and the like. The systems and methods described herein may be configured to analyze the overall popularity of a drug, and apply different scores to seasonal drug, critical therapy drug, iced medication, controlled drug, and maintenance drug. The systems and methods described herein may be configured to determine weight values based on a prescribing healthcare professional response rates for the drug as well as the drug-patient combination.

In some embodiments, the systems and methods described herein may be configured to rate patients by a likelihood of life time value in terms of the patient commitment to using prescription delivery services, while converting at least some of the patient prescriptions to mail delivery (e.g., and having less long term financial cost to the high volume pharmacy or the insurance provider). The systems and methods described herein may be configured to consider patient personality traits and patterns of purchase to determine contributing factors of losing the patient over time. The systems and methods described herein may be configured to provide a weighting system as an option for the management to change the optimized weights depending on various preferences (e.g., cost margin may be weighted differently than a tenure of the patient). The systems and methods described herein may be configured to provide a customized mathematical algorithm that uses the weighting factors (e.g., cost minimization+patient satisfaction), while taken into account the constraints with scores received from artificial intelligence models for each layer of prescriber, drug, and patient. The systems and methods described herein may be configured to choose scoring weights, which may be based on the demand and population type.

As described, the systems and methods described herein may be configured to use a mathematical model for a first layer of analysis (e.g., which may be referred to herein as Layer 1 or the Prescriber Layer). The mathematical model may be defined according to:
pϵP: Prescriber p in the list of all prescribers, P
Nₚ: Total number of prescriber
nₚ: Number of times contacted the prescriber p, historically
rₚ: Probability of prescriber p responses to a communication
tₚ: Historical turnaround response days for prescriber p
cₚ: Probabilty of historical communication channel response for prescriber p

The k-nearest neighborhood algorithm is used to find the categories for the response rate and turn-around days. For instance, response rate of more than 90% and turn-around days of less than 4 days receive the highest weights for wᵣ_{ₚ} and wₜ_{ₚ}·wᵣ_{ₚ} is based on the business needs and result of the k, nearest neighborhood, the weight assigned to the prescriber p's response rate, wₜ_{_{P}}:

wₜ_{_{P}}: Based on the business needs and result of the k-nearest neighborhood, the weight assigned to the historical turnaround response days for prescriber p

w_{c}_{ₚ}: Based on the nₚ and cₚ, the right targeting channel needs to be assigned to the prescriber. It should be understood that, while the k-nearest neighborhood algorithm is referenced and described herein and throughout for illustrative purposes, any suitable clustering method can be used as a categorization technique.

If the variation in distribution of the prescriber's response over all channels is slim, a small weight is assigned to the parameter according to:

$$Normalize the weights until : max ( {w}_{rp} ) + max ( {w}_{tp} ) + max ( {w}_{cp} ) = 1$$

Accept the prescriber's data until enough samples are gathered for the prescriber such that:

$$if \{ \begin{matrix}{n}_{p} < 4 \\ {n}_{p} \geq 4 , assign {w}_{rp} , {w}_{tp} , and {w}_{cp} to ∀ p ∈ P\end{matrix} \\ {w}_{p} : Weight Assigned to prescriber p \\ {w}_{p} = ( {w}_{rp} ) + ( {w}_{tp} ) + ( {w}_{cp} ) , ∀ p ∈ P \\ 0 \leq {w}_{p} \leq 1 , ∀ p ∈ P$$

For sure populations, assign different sub-weights to the age of the data. For example, more recent data may receive the highest weight, while the most historical data receives the lowest weight.

The systems and methods described herein may be configured to use the mathematical model for a second layer of analysis (e.g., which may be referred to herein as Layer 2 or the Drug Layer). The mathematical model may be further defined for the second layer according to:
dϵD: Drug d in a list of all maintenace Rxs, D
N: Total number of prescriber
n_{pd}: Numberof times prescriber p accepted drug d
r_{pd}: Probability of prescriber p responses to drug d
r_{d}: Rank of historical rate of approval for drug d over all prescribers P and their frequencies over time
r_{dt}: Rank of historical rate of approval for certain therapeutic index over all historical data

The k-nearest neighborhood algorithm is used to find the ranges and categories for r_{pd},

d, and rat.

w_{pd}: Based on the business needs and result of the k-nearest neighborhood, the weight assigned to the drug d approved by prescriber p

w_{d}: Based on the business needs and result of the k-nearest neighborhood, the weight assigned to the drug d

w_{dt}: Based on the business needs and result of the k-nearest neighborhood, the weight assigned to the therapeutic index t

$$Normalize the weights until : max ( {w}_{pd} ) + max ( {w}_{d} ) + max ( {w}_{dt} ) = 1$$

Accept the data until enough samples are gathered for drug d and prescriber p:

$$if \{ \begin{matrix}{n}_{p} < 4 \\ {n}_{pd} \geq 4 , assign {w}_{pd} , {w}_{d} , and {w}_{dt} to ∀ p ∈ P , d ∈ D\end{matrix}$$

For these populations, assign different sub-weights based on the age of the data. For example, more recent data may receive higher weight value, while the most historical data receives the lowest weight value, which may be defined according to:

$${w}_{d} : Weight Assigned to drug d \\ {w}_{d} = ( {w}_{pd} ) + ( {w}_{d} ) + ( {w}_{dt} ) , ∀ p ∈ P , d ∈ D \\ 0 \leq {w}_{d} \leq 1 , ∀ p ∈ P , d ∈ D$$

The systems and methods described herein may be configured to use the mathematical model for a second layer of analysis (e.g., which may be referred to herein as Layer 2 or the Drug Layer). The mathematical model may be further defined for the second layer according to:

ptϵPT: Patient pt in the list of all eligible first time home delivery patients with maintenance drug, PT N_{PT}: Total number of patients

The systems and methods described herein may be configured to use Kendall rank correlation and Spearman correlation techniques to view the strengths and correlations of the following parameters against patient targeting success in terms of the duration of a patient tenure as well as conversation of other medications in household, Such ranking may use parameters, such as, for example, only: Age range, w_{PT}_{_{A}}: Depending on the age range, strength of correlation varies; Gender, w_{PT}_{_{G}}: Men have higher chance to churn: Probability of loyalty (Tenure), w_{PT}_{ₜ}: How long a member has been with ESI and number of times renewed the membership; Urban/Rural, +/−w_{PT}_{_{UR}}: Probability of Rural members being loyal is higher; Member's family size, W_{PT}_{_{f}}: More opportunity size with larger families and medicine cabinet size; Disease state, W_{PT}_{_{d}}: Duration of commitment after conversion; Number of times churned in the past, W_{PT}_{_{ch}}: Member being consistence with their decision or more time/cost from agents, procedures, etc.; Number of in-bound initiative that patients had with ESI agent to make a conversion, W_{PT}_{_{conv}}; Member's maintenance cost such as the number of monthly communication with ESI agent for every drug, W_{PT}_{_{comm}}; Medicine cabinet size and probability of conversion to home delivery,

$${w}_{{PT}_{{c}_{m}}} ;$$

Current target drug's margin,

$${w}_{{PT}_{{c}_{d}}}$$

; Opportunity sizeu maigin based on the disease state and household's medicine cabinet size,

$${w}_{{PT}_{{c}_{f}}}$$

; and/or any other suitable parameter.

The systems and methods described herein may be configured to use a Random Forest Regressor (RFR) technique to score the above parameters based on identified importance on a life time value of a patient to the high volume pharmacy and/or the insurance provider. Depending on the business needs, more parameters can be added to this model. These scores, which sum up to 1, are used as weights on each patient. The members with higher weight are more likely to accept the retail to mail conversion as well as being a loyal member.

The systems and methods described herein may be configured to, if data is not available for some of the above parameters, assign value of zero to the weights of missing parameters and the RFR is computed to obtain appropriate scores for the available data. As more information is gathered on the patient, the scoring model can be updated according to:

$${w}_{{PT}_{A}} + {w}_{{PT}_{G}} + {w}_{{PT}_{t}} + {w}_{{PT}_{ur}} + {w}_{{PT}_{f}} + {w}_{{PT}_{d}} + {w}_{{PT}_{ch}} + {w}_{{PT}_{conv}} + {w}_{{PT}_{comm}} + {w}_{{PT}_{{c}_{m}}} + {w}_{{PT}_{{c}_{d}}} + {w}_{{PT}_{{c}_{f}}} = 1 \\ {w}_{pt} : Weight Assigned to patient pt \\ {w}_{pt} = {w}_{{PT}_{A}} + {w}_{{PT}_{G}} + {w}_{{PT}_{t}} + {w}_{{PT}_{ur}} + {w}_{{PT}_{f}} + {w}_{{PT}_{d}} + {w}_{{PT}_{ch}} + {w}_{{PT}_{conv}} + {w}_{{PT}_{comm}} + {w}_{{PT}_{{c}_{m}}} + {w}_{{PT}_{{c}_{d}}} + {w}_{{PT}_{{c}_{f}}} , ∀ p ∈ P , d ∈ D , pt ∈ PT$$

In some embodiments, the systems and methods described herein may be configured to, based on the defined business needs of the high volume pharmacy and/or the insurance provide, assign additional level of weight to each of Layer 1, wₚ_{ₗ₂}, Layer 2, w_{d}_{ₗ₂} and Layer 3, wₚₜ_{ₗ₂}. This may result in:

$${w}_{{p}_{{l}_{2}}} + {w}_{{d}_{{l}_{2}}} + {w}_{{pt}_{{l}_{2}}} = 1 , ∀ p ∈ P , d ∈ D , pt ∈ PT$$

The systems and methods described herein may be configured to determine the targeting list using the result of the following score for each prescriber, drug, and patient combination. The list is populated based on the highest to lowest scores as well as the resource budget available for agents, according to:

$${w}_{p} * {w}_{{p}_{{l}_{2}}} + {w}_{d} * {w}_{{d}_{{l}_{2}}} + {w}_{pt} * {w}_{{pt}_{{l}_{2}}} = 1 , ∀ p ∈ P , d ∈ D , pt ∈ PT$$

In some embodiments, the systems and methods described herein may be configured to train a machine learning model (e.g., which may include features similar to the mathematical model and/or additional features and may be generated by an artificial intelligence engine) using data from a large infrastructure environment, where the model learns from mistakes. The systems and methods described herein may be configured to not eliminate new prescribing healthcare professionals will from the filters until enough of a sample size on a pattern of behavior is received from by the model. The systems and methods described herein may be configured to use, for new members, first demographics and/or lifestyle features to find the likelihood of churn. The systems and methods described herein may be configured to add the features to analyze the patient (e.g. or member) as more information is gathered for respective members. The systems and methods described herein may be configured to provide a toolbox, which may be integrated into different datasets.

In some embodiments, the systems and methods described herein may be configured to merge, combine, and automate an artificial intelligences engine and/or the machine learning model that is written using a suitable statistical programming tool with many different queries that extract data from a database server, data, and claims to score the supply chain weekly target list. The systems and methods described herein may be configured to use three summary lookup tables to take into account the information on pattern and behavior of prescribers, drugs, and therapy. These look up tables may be refreshed periodically (e.g., monthly or other suitable period). For example, once weekly, when the target list is finalized by the supply chain team, the systems and methods described herein may be configured to may create a temporarily table. This table contains a copy of the weekly target information along with the historical information obtained from the monthly lookup tables on prescribers, drugs, and therapies. The systems and methods described herein may be configured to add additional parameters on patient demographic and medicine cabinet to the temporarily table. The systems and methods described herein may be configured to input the information for the machine learning model in the suitable statistical programming tool. The systems and methods described herein may be configured to score, using the suitable statistical programming tool each row of data based on the probability of success and expected margin. The systems and methods described herein may be configured to use a primary key to identify the patient, prescriber, and drug, along with the target score, which may be stored in a permanent table.

In some embodiments, the systems and methods described herein may be configured to identify patients with higher likelihood to respond to outreach by the high volume pharmacy and/or the insurance provider, such as patients consuming a relatively large volume of medications and are expecting a conversation with the pharmacy or those who historically have responded to campaigns via a preferred channel of communication.

In some embodiments, the systems and methods described herein may be configured to read campaign input files associated with a campaign and load to campaign tables for consumption downstream. A campaign may include a member communication. The systems and methods described herein may be configured to analyze data and target an audience for a communication (e.g., an electronic mail, text message, postal mail, telephone, and the like). The input files may be business unit specific and may define product (e.g., drugs or other prescription products or product accessories) exclusions and inclusions.

In some embodiments, the systems and methods described herein may be configured to pull claims based on different campaign products (e.g., mandatory products, voluntary products, and other products). The systems and methods described herein may be configured to combine all product claims and create dynamic campaign tables. The systems and methods described herein may be configured to add client enrollment attributes. For example, the systems and methods described herein may be configured to retrieve claims data including mandatory claims dada, voluntary claims data, and the like. The claims data may indicate information associated with prescribing healthcare professionals, members, and products.

In some embodiments, the systems and methods described herein may be configured to run campaign global and/or claim level suppression edits and load drop records to the campaign drop tables. The systems and methods described herein may be configured to clean data to remove records that should be excluded (e.g., data associated with a member terming out or other suitable data to exclude). The suppression of data may be defined by a functional requirement document associated with the high volume pharmacy and/or the insurance provider. The systems and methods described herein may be configured to load remaining records that survive the suppression edits for channel specific processing downstream.

In some embodiments, the systems and methods described herein may be configured to send fill pharmacy addresses to a Geo-Location system to request alternate pharmacies (e.g., pharmacies within a defined geographic distance of a fill pharmacy). The systems and methods described herein may be configured to send voluntary product claims to the Pricing system to request drug pricing. The systems and methods described herein may be configured to use the drug pricing to calculate potential member savings for alternative drugs or alternative purchase locations for drugs (e.g., associated with a prescription or over the counter).

In some embodiments, the systems and methods described herein may be configured to generate output for an electronic mail campaign. For example, the systems and methods described herein may be configured to process alternate pharmacy results from the Geo-Location system, select appropriate alternate pharmacies based on an in-network pharmacy list associated with a respective member and select the top alternate pharmacies (e.g., top 3 or other suitable number of alternatively pharmacies). The systems and methods described herein may be configured to calculate member and client drug savings. The systems and methods described herein may be configured to run campaign electronic mail suppression edits and keep record that are appropriate for electronic mail communication. The systems and methods described herein may be configured to assign electronic mail channel version identification information and fulfillment attributes based on unique product combinations associated with the respective member. The systems and methods described herein may be configured to generate an electronic mail target list. The target list may be used to automatically generate electronic mail (e.g. or text messages or other electronic communications) messages and send the messages to the respective members.

The systems and methods described herein may be configured to roll multiple communications for a member into single communication. The systems and methods described herein may be configured to provide a versioning code to the file so a future system can read it and translate to know what products were included. The systems and methods described herein may be configured to save the target list and/or the messages to an electronic mail table.

In some embodiments, the systems and methods described herein may be configured to generate a postal mail targeting list for a postal mail communication campaign. The systems and methods described herein may be configured to run campaign mail suppression edits and keep records that are appropriate for mail communication. The systems and methods described herein may be configured to assign mail channel version identification information and fulfillment attributes based on unique product combinations of respective members. The systems and methods described herein may be configured to create a mail target list.

In some embodiments, the systems and methods described herein may be configured to generate a target list for a telephone communication campaign. The systems and methods described herein may be configured to run campaign telephone suppression edits and keep records that are appropriate for telephone communication. The systems and methods described herein may be configured to assign telephone channel version identification information and fulfillment attributes based on unique product combinations of respective members. The systems and methods described herein may be configured to create telephone target list. The systems and methods described herein may be configured to generate campaign quality check (QC) report. The systems and methods described herein may be configured to send electronic mail, postal mail, and telephone target lists to a communication platform for fulfillment.

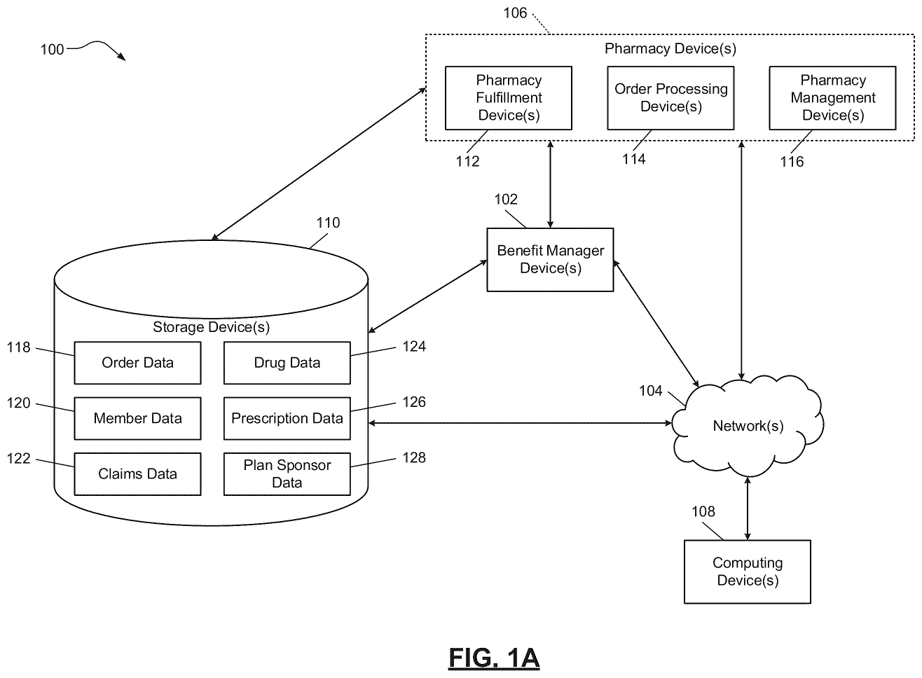

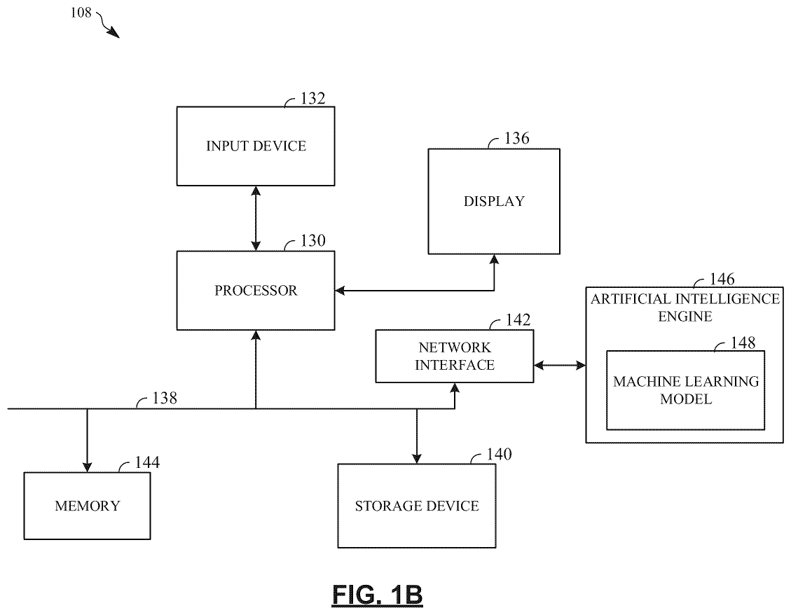

FIG. 1A is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104. The system 100 may also include a storage device 110.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in the storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfilment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the use of the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan-for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

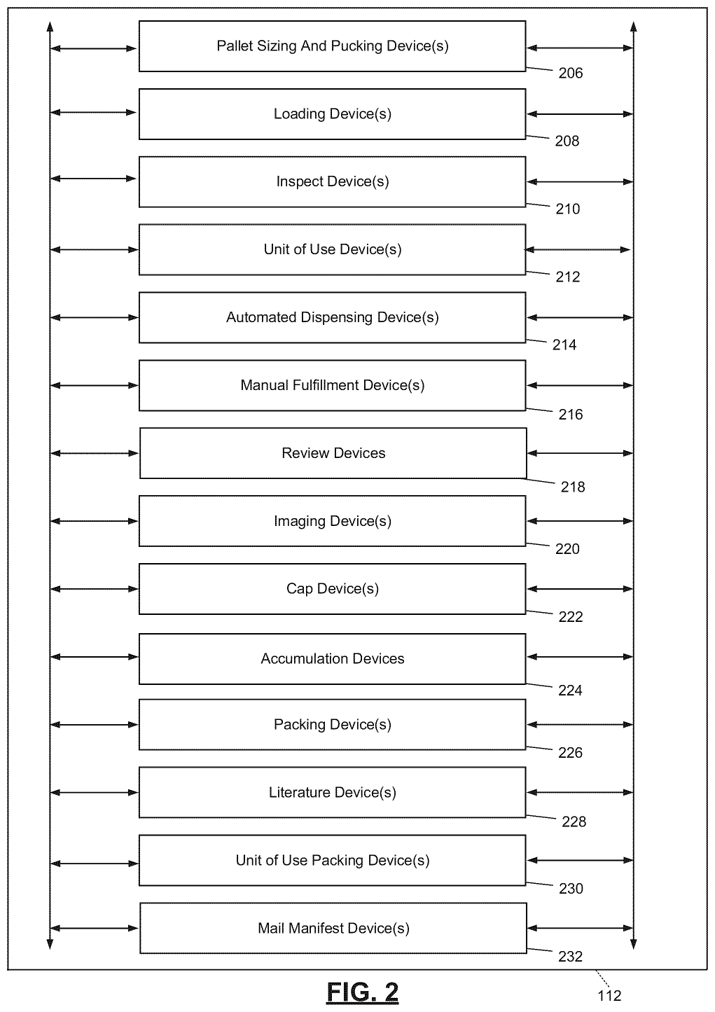

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

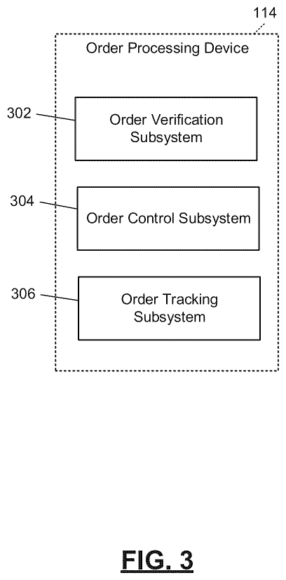

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may include order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, and the like. The order tracking subsystem 306 may store data locally (for example, in a memory) or as a portion of the order data 118 stored in the storage device 110.

In some embodiments, the system 100 may include one or more computing devices 108, as is generally illustrated in FIG. 1B. The computing device 108 may include any suitable computing device, such as a mobile computing device, a desktop computing device, a laptop computing device, a server computing device, other suitable computing device, or a combination thereof. The computing device 108 may be used by a user accessing the pharmacy associated with the system 100, as described. Additionally, or alternatively, the computing device 108 may be configured to identify an optimum or substantially optimum combination of data objects, as described.

The computing device 108 may include a processor 130 configured to control the overall operation of computing device 108. The processor 130 may include any suitable processor, such as those described herein. The computing device 108 may also include a user input device 132 that is configured to receive input from a user of the computing device 108 and to communicate signals representing the input received from the user to the processor 130. For example, the user input device 132 may include a button, keypad, dial, touch screen, audio input interface, visual/image capture input interface, input in the form of sensor data, etc.

The computing device 108 may include a display 136 that may be controlled by the processor 130 to display information to the user. A data bus 138 may be configured to facilitate data transfer between, at least, a storage device 140 and the processor 130. The computing device 108 may also include a network interface 142 configured to couple or connect the computing device 108 to various other computing devices or network devices via a network connection, such as a wired or wireless connection, such as the network 104. In some embodiments, the network interface 142 includes a wireless transceiver.

The storage device 140 may include a single disk or a plurality of disks (e.g., hard drives), one or more solid-state drives, one or more hybrid hard drives, and the like. The storage device 140 may include a storage management module that manages one or more partitions within the storage device 140. In some embodiments, storage device 140 may include flash memory, semiconductor (solid state) memory or the like. The computing device 108 may also include a memory 144. The memory 144 may include Random Access Memory (RAM), a Read-Only Memory (ROM), or a combination thereof. The memory 144 may store programs, utilities, or processes to be executed in by the processor 130. The memory 144 may provide volatile data storage, and stores instructions related to the operation of the computing device 108.

In some embodiments, the computing device 108 may use an artificial intelligence engine 146 configured to use at least one machine learning model 148 to perform the embodiments of systems and methods described herein. The artificial intelligence engine 146 may include any suitable artificial intelligence engine and may be disposed on computing device 108 or remotely located from the computing device 108, such as in a cloud computing device or other suitable remotely located computing device. The artificial intelligence engine 146 may use one or more machine learning models 148 to perform at least one of the embodiments disclosed herein. The computing device 108 may include a training engine capable of generating the one or more machine learning models 148. The machine learning models 148 may be trained to identify prescribing healthcare professional, product, and member combinations and/or to identify target members for communication.

In some embodiments, the processor 130 may be configured to execute instructions stored on the memory 144 that cause the processor 130 and/or the computing device 108 to receive prescription data associated with a plurality of prescriptions written by a prescribing healthcare professional. The prescription data may include prescription product data associated with one or more prescriptions of the prescription data, prescriber data associated the prescribing healthcare professional, member identification data associated with the one or more prescriptions of the prescription data, and/or any other suitable data. The prescription product data may include prescription approval rate data for prescription products of the prescription product data, prescription turnaround time data for prescription products of the prescription product data, and/or any other suitable data.

The computing device 108 may determine a prescriber score for the prescribing healthcare professional based on the prescription data. The computing device 108 may determine a prescription product score for each prescription product in each respective prescription of the plurality of prescriptions associated with the prescription data. Each prescription product may include at least one of a prescription drug or a prescription accessory. The prescription drug may include any suitable drug. The prescription accessory may include any suitable prescription accessory, such as syringes, pads, bandages, cleaners, documentation, and/or any other suitable accessory. The prescription product score for a respective prescription product may be determined based on an elapsed period between a request for refilling a prescription associated with the respective prescription product and the prescribing healthcare professional writing responding to the request, a therapeutic index of the respective prescription product, an historical approval rate for the respective prescription product by all prescribing healthcare professionals, an historical approval rate for the respective prescription product by the prescribing healthcare professional, any other suitable information, or a combination thereof.

The computing device 108 may retrieve, from a member database (e.g., which may be stored on or associated with the storage device 110), member data associated with respective members of a plurality of members represented in the member database. The respective members may have a treatment association with the prescribing healthcare professional. The computing device 108 may determine a member score for each respective member of the plurality of members based on at least one home delivery factor. The at least one home delivery factor may at least one of a digital inclination (e.g., or a member to engage with digital tools, such as computing device, including those described herein), a disease state (e.g., of the member), a market segment, a geographic location (e.g., relative to local pharmacies and a distance between the home of a member and the closest pharmacy), and other suitable factor, or a combination thereof.

The computing device 108 may determine, based on the prescriber score, each prescription product score, and each member score, and using the artificial intelligence engine 146 configured to use at least one machine learning model 148, a prescriber propensity score for the prescribing healthcare professional. The computing device 108 may, in response to a determination that the prescriber propensity score is greater than a first threshold, identify respective members having a member score that is greater than a second threshold, and generate, for display, an output indicating at least the prescriber propensity score and a list of identified respective members.

In some embodiments, the computing device 108 may receive a plurality of prescriber scores for respective prescribing healthcare professionals. The computing device 108 may receive a plurality of prescription product scores for respective prescription products. The computing device 108 may receive a plurality of member scores for receptive members. Each respective member being may be associated with at least one of at least one prescription product associated with a prescription product score of the plurality of prescription product scores and at least one prescribing healthcare professional associated with a prescriber score of the plurality of prescriber scores.

The computing device 108 may receive targeted communication input indicating a prescriber score weight, a prescription product score weight, and a member score weight. The computing device 108 may identify at least one combination of a prescribing healthcare professional, a prescription product, and a member using the artificial intelligence engine 146 configured to use the at least one machine learning model 148 configured to use the prescriber score, the prescriber score weight, the prescription production score, the prescription product score weight, the member score, and the member score weight to predict the at least one combination. The machine learning model may be trained using any suitable data and may receive feedback from the computing device 108 and/or a user indicating an accuracy of the predictions of the combinations. The machine learning model 148 may be subsequently trained using the feedback. The computing device 108 may generate, for display at the display 136 or other suitable display, an output indicating at least the at least one combination of a prescribing healthcare professional, a prescription product, and a member.

In some embodiments, the computing device 108 may receive a plurality of member communication campaign input files that define a set of pharmacy product exclusion rules and a set of pharmacy product inclusion rules. The plurality of member communication campaign input files may be associated with a member communication campaign. The pharmacy product exclusion rules may define or identify pharmacy products to exclude from the campaign. The pharmacy product inclusion rule may define or identify pharmacy products to include in the campaign.

The computing device 108 may generate one or more campaign tables in a campaign table database (e.g., which may be stored on or associated with the storage device 110) based the set of pharmacy product exclusion rules and the set of pharmacy product inclusion rules. The computing device 108 may retrieve, from an insurance claim database (e.g., which may be stored on or associated with the storage device 110) and based on the plurality of member communication campaign input files, insurance claim data. The insurance claim data may include member data. The member data may include at least member identification data, member enrollment status data, prescription product data, pharmacy location data, pharmacy identification data, and/or other suitable data.

The computing device 108 may populate records of the one or more campaign tables with the insurance claim data. The computing device 108 may suppress data in the one or more campaign tables based on a set of suppression rules. The set of suppression rues may be associated with a function requirement document associated with at least one of the high volume pharmacy and the insurance provider. The member may be associated with the high volume pharmacy (e.g. a user or potential user of the high volume pharmacy), and/or the insurance provider. The commuting device 108 may remove the suppressed data from the one or more campaign tables. The computing device 108 may load the suppressed data in a drop table of the campaign tables database.

The computing device 108 may, for at least one record of the one or more campaign tables, identify a pharmacy used by an associated member to fill one or more prescriptions. The computing device 108 may identify one or more alternative pharmacies within a predetermined distance from the identified pharmacy. The computing device 108 may identify the one or more alternative pharmacies within a predetermined distance from the identified pharmacy by communicating an address associated with the identified pharmacy to a geo-location identification system and receiving, from the geo-location identification system, the one or more alternative pharmacies. The at least one alternative pharmacy criteria may indicate that the at least one alternative pharmacy is in network (e.g., for the member according to an insurance policy of the member).

The computing device 108 may identify at least one product of the one or more prescriptions. The computing device 108 may identify one or more alternative products associated with the at least one product of the one or more prescriptions. The computing device 108 may generate a communication data object that includes (i) at least one alternative pharmacy of the one or more alternative pharmacies that meet at least one alternative pharmacy criteria and (ii) at least one alternative product of the one or more alternative products that meet at least one alternative product criteria. The at least one alternative product criteria may indicate that the at least one alternative product is a therapeutic alternative for the at least one product of the one or more prescriptions.

The computing device 108 may communicate the communication data object to the associated member. The computing device 108 may use the communication data object to generate electronic mail messages, to generate postal mail messages, and/or to generate telephonic messages or calls. Additionally, or alternatively, the computing device 108 may transmit the communication data object to another system to generate corresponding campaign messages.

In some embodiments, the computing device 108 and/or the system 100 may perform the methods described herein. However, the methods described herein as performed by the computing device 108 and/or the system 100 are not meant to be limiting, and any type of software executed on a computing device or a combination of various computing devices can perform the methods described herein without departing from the scope of this disclosure.

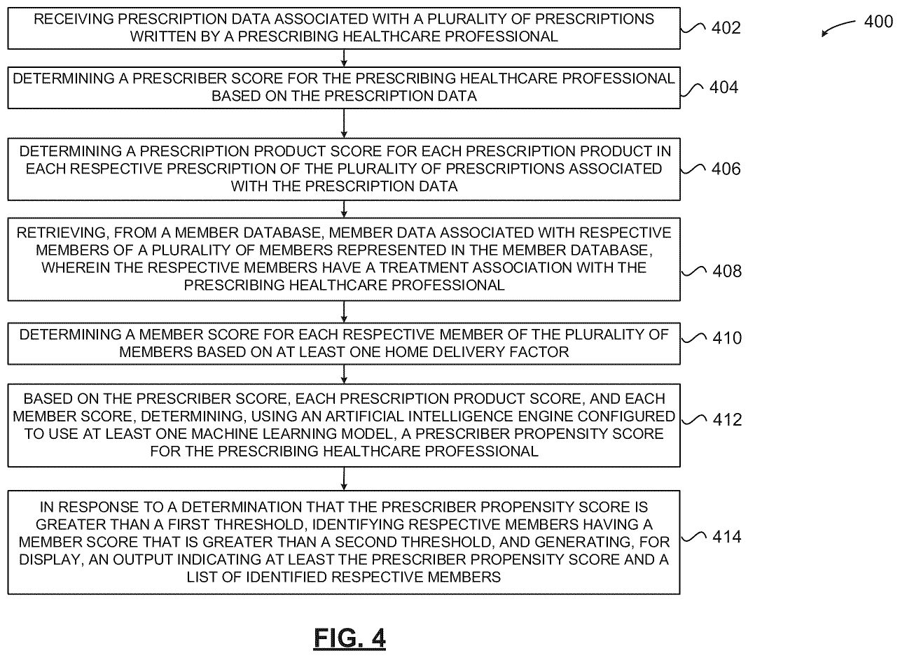

FIG. 4 is a flow diagram generally illustrating a prescriber home delivery propensity determination method 400 according to the principles of the present disclosure. At 402, the method 400 receives prescription data associated with a plurality of prescriptions written by a prescribing healthcare professional. For example, the computing device 108 may receive the prescription data associated with the plurality of prescriptions written by the prescribing healthcare professional.

At 404, the method 400 determines a prescriber score for the prescribing healthcare professional based on the prescription data. For example, the computing device 108 may determine the prescriber score for the prescribing healthcare professional based on the prescription data.

At 406, the method 400 determines a prescription product score for each prescription product in each respective prescription of the plurality of prescriptions associated with the prescription data. For example, the computing device 108 may determine the prescription product score for each prescription product in each respective prescription of the plurality of prescriptions associated with the prescription data.

At 408, the method 400 retrieves, from a member database, member data associated with respective members of a plurality of members represented in the member database. The respective members may have a treatment association with the prescribing healthcare professional. For example, the computing device 108 may retrieve, from the member database, member data associated with respective members of a plurality of members represented in the member database.

At 410, the method 400 determines a member score for each respective member of the plurality of members based on at least one home delivery factor. For example, the computing device 108 may determines the member score for each respective member of the plurality of members based on the at least one home delivery factor.

At 412, the method 400, based on the prescriber score, each prescription product score, and each member score, determines, using an artificial intelligence engine configured to use at least one machine learning model, a prescriber propensity score for the prescribing healthcare professional. For example, the computing device 108 may, based on the prescriber score, each prescription product score, and each member score, determine, using the artificial intelligence engine 146 configured to use the at least one machine learning model 148, the prescriber propensity score for the prescribing healthcare professional.

At 414, the method 400, in response to a determination that the prescriber propensity score is greater than a first threshold, identifies respective members having a member score that is greater than a second threshold, and generates, for display, an output indicating at least the prescriber propensity score and a list of identified respective members. For example, the computing device 108 may, in response to the determination that the prescriber propensity score is greater than the first threshold, identifies respective members having the member score that is greater than the second threshold, and generates, for display, the output indicating at least the prescriber propensity score and the list of identified respective members.

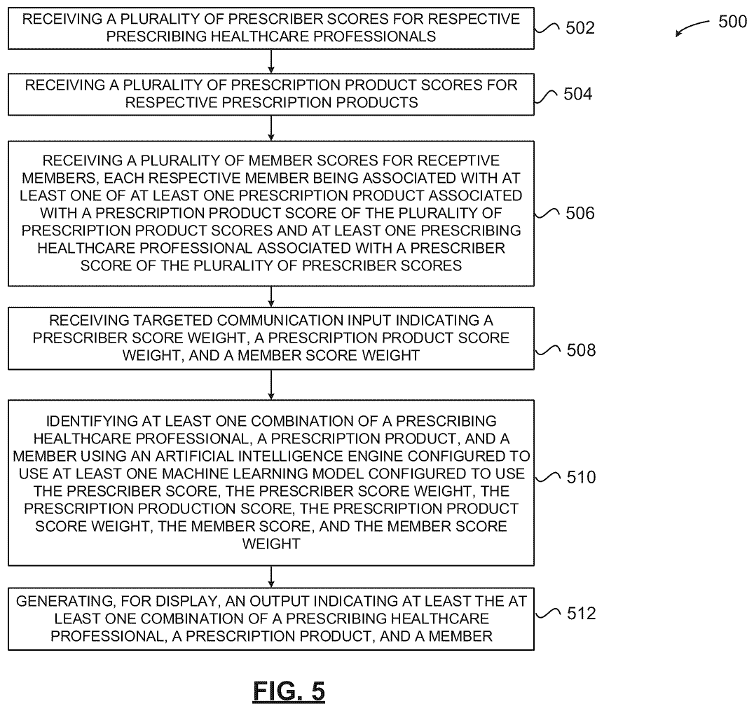

FIG. 5 is a flow diagram generally illustrating a member targeting method 500 according to the principles of the present disclosure. At 502, the method 500 receives a plurality of prescriber scores for respective prescribing healthcare professionals. For example, the computing device 108 may receive the plurality of prescriber scores for the respective prescribing healthcare professionals.

At 502, the method 500 receives a plurality of prescription product scores for respective prescription products. For example, the computing device 108 may receive the plurality of prescription product scores for the respective prescription products.

At 504, the method 500 receives a plurality of member scores for receptive members. Each respective member may be associated with at least one of at least one prescription product associated with a prescription product score of the plurality of prescription product scores and at least one prescribing healthcare professional associated with a prescriber score of the plurality of prescriber scores. For example, the computing device 108 may receive the plurality of member scores for the receptive members.

At 506, the method 500 receives targeted communication input indicating a prescriber score weight, a prescription product score weight, and a member score weight. For example, the computing device 108 may receive the targeted communication input indicating the prescriber score weight, the prescription product score weight, and the member score weight.

At 508, the method 500 identifies at least one combination of a prescribing healthcare professional, a prescription product, and a member using an artificial intelligence engine configured to use at least one machine learning model configured to use the prescriber score, the prescriber score weight, the prescription production score, the prescription product score weight, the member score, and the member score weight. For example, the computing device 108 may identify the at least one combination of the prescribing healthcare professional, the prescription product, and the member using the artificial intelligence engine 146 configured to use the at least one machine learning model 148 configured to use the prescriber score, the prescriber score weight, the prescription production score, the prescription product score weight, the member score, and the member score weight.

At 510, the method 500 generates, for display, an output indicating at least the at least one combination of a prescribing healthcare professional, a prescription product, and a member. For example, the computing device 108 may generate, for display, the output indicating at least the at least one combination of the prescribing healthcare professional, the prescription product, and the member.

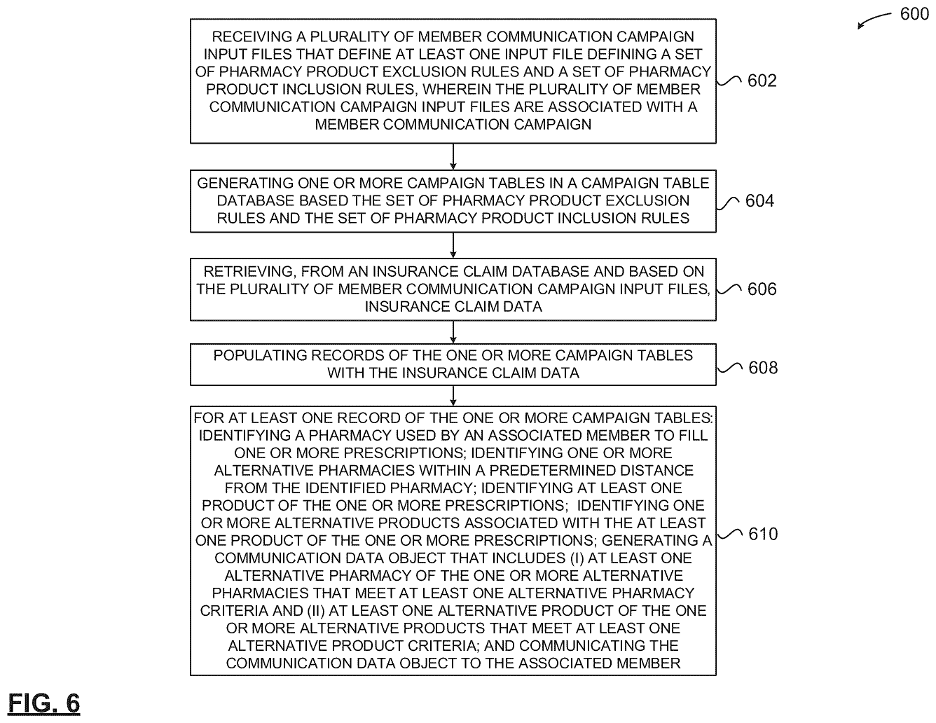

FIG. 6 is a flow diagram generally illustrating a member communication generation method 600 according to the principles of the present disclosure. At 602, the method 600 receives a plurality of member communication campaign input files that define a set of pharmacy product exclusion rules and a set of pharmacy product inclusion rules. The plurality of member communication campaign input files may be associated with a member communication campaign. For example, the computing device 108 may receive the plurality of member communication campaign input files that define the set of pharmacy product exclusion rules and the set of pharmacy product inclusion rules.

At 604, the method 600 generates one or more campaign tables in a campaign table database based the set of pharmacy product exclusion rules and the set of pharmacy product inclusion rules. For example, the computing device 108 may generate the one or more campaign tables in the campaign table database based the set of pharmacy product exclusion rules and the set of pharmacy product inclusion rules.

At 606, the method 600 retrieves, from an insurance claim database and based on the plurality of member communication campaign input files, insurance claim data. For example, the computing device 108 may retrieve, from the insurance claim database and based on the plurality of member communication campaign input files, the insurance claim data.

At 608, the method 600 populates records of the one or more campaign tables with the insurance claim data. For example, the computing device 108 may populate records of the one or more campaign tables with the insurance claim data.

At 610, the method 600 suppresses data in the one or more campaign tables based on a set of suppression rules. For example, the computing device 108 may suppress the data in the one or more campaign tables based on the set of suppression rules.

At 612, the method 600, for at least one record of the one or more campaign tables, identifies a pharmacy used by an associated member to fill one or more prescriptions. The method 600 may identify one or more alternative pharmacies within a predetermined distance from the identified pharmacy. The method 600 may identify at least one product of the one or more prescriptions. The method 600 may identify one or more alternative products associated with the at least one product of the one or more prescriptions. The method 600 may generate a communication data object that includes (i) at least one alternative pharmacy of the one or more alternative pharmacies that meet at least one alternative pharmacy criteria and (ii) at least one alternative product of the one or more alternative products that meet at least one alternative product criteria. The method 600 may communicate the communication data object to the associated member. For example, the computing device 108 may, for the at least one record of the one or more campaign tables, identify the pharmacy used by the associated member to fill the one or more prescriptions. The computing device 108 may identify the one or more alternative pharmacies within the predetermined distance from the identified pharmacy. The computing device 108 may identify the at least one product of the one or more prescriptions. The computing device 108 may identify the one or more alternative products associated with the at least one product of the one or more prescriptions. The computing device 108 may generate the communication data object that includes (i) the at least one alternative pharmacy of the one or more alternative pharmacies that meet at least one alternative pharmacy criteria and (ii) the at least one alternative product of the one or more alternative products that meet at least one alternative product criteria. The computing device 108 may communicate the communication data object to the associated member.

Clause 1. A system for determining a propensity of a prescribing healthcare professional to write a prescription for home delivery, the system comprising: a processor; and a memory including instructions that, when executed by the processor, cause the processor to: receive prescription data associated with a plurality of prescriptions written by a prescribing healthcare professional; determine a prescriber score for the prescribing healthcare professional based on the prescription data; determine a prescription product score for each prescription product in each respective prescription of the plurality of prescriptions associated with the prescription data; retrieve, from a member database, member data associated with respective members of a plurality of members represented in the member database, wherein the respective members have a treatment association with the prescribing healthcare professional; determine a member score for each respective member of the plurality of members based on at least one home delivery factor; based on the prescriber score, each prescription product score, and each member score, determine, using an artificial intelligence engine configured to use at least one machine learning model, a prescriber propensity score for the prescribing healthcare professional; and, in response to a determination that the prescriber propensity score is greater than a first threshold: identify respective members having a member score that is greater than a second threshold; and generate, for display, an output indicating at least the prescriber propensity score and a list of identified respective members.

Clause 2. The system of any clause herein, wherein the prescription data includes prescription product data associated with one or more prescriptions of the prescription data, prescriber data associated the prescribing healthcare professional, and member identification data associated with the one or more prescriptions of the prescription data.

Clause 3. The system of any clause herein, wherein the prescription product data includes at least prescription approval rate data for prescription products of the prescription product data and prescription turnaround time data for prescription products of the prescription product data.

Clause 4. The system of any clause herein, wherein each prescription product includes at least one of a prescription drug or a prescription accessory.

Clause 5. The system of any clause herein, wherein the prescription product score for a respective prescription product is based on an elapsed period between a request for refilling a prescription associated with the respective prescription product and the prescribing healthcare professional writing responding to the request.

Clause 6. The system of any clause herein, wherein the prescription product score for a respective prescription product is based on a therapeutic index of the respective prescription product.

Clause 7. The system of any clause herein, wherein the prescription product score for a respective prescription product is based on at least one of an historical approval rate for the respective prescription product by all prescribing healthcare professionals and an historical approval rate for the respective prescription product by the prescribing healthcare professional.

Clause 8. The system of any clause herein, wherein the at least one home delivery factor includes at least one of a digital inclination, a disease state, a market segment, and a geographic location.

Clause 9. A method for determining a propensity of a prescribing healthcare professional to write a prescription for home delivery, the method comprising: receiving prescription data associated with a plurality of prescriptions written by a prescribing healthcare professional; determining a prescriber score for the prescribing healthcare professional based on the prescription data; determining a prescription product score for each prescription product in each respective prescription of the plurality of prescriptions associated with the prescription data; retrieving, from a member database, member data associated with respective members of a plurality of members represented in the member database, wherein the respective members have a treatment association with the prescribing healthcare professional; determining a member score for each respective member of the plurality of members based on at least one home delivery factor; based on the prescriber score, each prescription product score, and each member score, determining, using an artificial intelligence engine configured to use at least one machine learning model, a prescriber propensity score for the prescribing healthcare professional; and, in response to a determination that the prescriber propensity score is greater than a first threshold: identifying respective members having a member score that is greater than a second threshold; and generating, for display, an output indicating at least the prescriber propensity score and a list of identified respective members.

Clause 10. The method of any clause herein, wherein the prescription data includes prescription product data associated with one or more prescriptions of the prescription data, prescriber data associated the prescribing healthcare professional, and member identification data associated with the one or more prescriptions of the prescription data.

Clause 11. The method of any clause herein, wherein the prescription product data includes at least prescription approval rate data for prescription products of the prescription product data and prescription turnaround time data for prescription products of the prescription product data.

Clause 12. The method of any clause herein, wherein each prescription product includes at least one of a prescription drug or a prescription accessory.

Clause 13. The method of any clause herein, wherein the prescription product score for a respective prescription product is based on an elapsed period between a request for refilling a prescription associated with the respective prescription product and the prescribing healthcare professional writing responding to the request.

Clause 14. The method of any clause herein, wherein the prescription product score for a respective prescription product is based on a therapeutic index of the respective prescription product.

Clause 15. The method of any clause herein, wherein the prescription product score for a respective prescription product is based on at least one of an historical approval rate for the respective prescription product by all prescribing healthcare professionals and an historical approval rate for the respective prescription product by the prescribing healthcare professional.

Clause 16. The method of any clause herein, wherein the at least one home delivery factor includes at least one of a digital inclination, a disease state, a market segment, and a geographic location.

Clause 17. An apparatus for determining a propensity of a prescribing healthcare professional to write a prescription for home delivery, the apparatus comprising: one or more processors; and at least one memory including instructions that, when executed by the one or more processors, cause the one or more processors, collectively or respectively, to: determine a prescriber score for a prescribing healthcare professional based on prescription data associated with a plurality of prescriptions written by the prescribing healthcare professional; determine a prescription product score for each prescription product in each respective prescription of the plurality of prescriptions associated with the prescription data; retrieve, from a member database, member data associated with respective members of a plurality of members represented in the member database, wherein the respective members have a treatment association with the prescribing healthcare professional; determine a member score for each respective member of the plurality of members based on at least one home delivery factor; based on the prescriber score, each prescription product score, and each member score, determine, using an artificial intelligence engine configured to use at least one machine learning model, a prescriber propensity score for the prescribing healthcare professional; and, in response to a determination that the prescriber propensity score is greater than a first threshold: identify respective members having a member score that is greater than a second threshold; and generate, for display, an output indicating at least the prescriber propensity score and a list of identified respective members.

Clause 18. The apparatus of any clause herein, wherein the prescription data includes prescription product data associated with one or more prescriptions of the prescription data, prescriber data associated the prescribing healthcare professional, and member identification data associated with the one or more prescriptions of the prescription data.

Clause 19. The apparatus of any clause herein, wherein the prescription product data includes at least prescription approval rate data for prescription products of the prescription product data and prescription turnaround time data for prescription products of the prescription product data.

Clause 20. The apparatus of any clause herein, wherein the prescription data is associated with a high volume pharmacy.

Clause 1.1 A system for identifying combinations of prescribing healthcare professionals, members, and products for targeted communication, the system comprising: a processor; and a memory including instructions that, when executed by the processor, cause the processor to: receive a plurality of prescriber scores for respective prescribing healthcare professionals; receive a plurality of prescription product scores for respective prescription products; receive a plurality of member scores for receptive members, each respective member being associated with at least one of at least one prescription product associated with a prescription product score of the plurality of prescription product scores and at least one prescribing healthcare professional associated with a prescriber score of the plurality of prescriber scores; receive targeted communication input indicating a prescriber score weight, a prescription product score weight, and a member score weight; identify at least one combination of a prescribing healthcare professional, a prescription product, and a member using an artificial intelligence engine configured to use at least one machine learning model configured to use the prescriber score, the prescriber score weight, the prescription production score, the prescription product score weight, the member score, and the member score weight; and generate, for display, an output indicating at least the at least one combination of a prescribing healthcare professional, a prescription product, and a member.

Clause 2.1 The system of any clause herein, wherein each prescribing score of the plurality of prescribing scores is determined based on prescription data associated with a plurality of prescriptions written by a respective prescribing healthcare professional.

Clause 3.1 The system of any clause herein, wherein the prescription data of a respective prescribing healthcare professional includes prescription product data associated with one or more prescriptions of the prescription data, prescriber data associated the respective prescribing healthcare professional, and member identification data associated with the one or more prescriptions of the prescription data.

Clause 4.1 The system of any clause herein, wherein the prescription product data includes at least prescription approval rate data for prescription products of the prescription product data and prescription turnaround time data for prescription products of the prescription product data.

Clause 5.1 The system of any clause herein, wherein each respective prescription product includes at least one of a prescription drug or a prescription accessory.

Clause 6.1 The system of any clause herein, wherein a prescription product score for a respective prescription product is based on an elapsed period between a request for refilling a prescription associated with the respective prescription product and a respective prescribing healthcare professional writing responding to the request.

Clause 7.1 The system of any clause herein, wherein a prescription product score for a respective prescription product is based on a therapeutic index of the respective prescription product.

Clause 8.1 The system of any clause herein, wherein a prescription product score for a respective prescription product is based on at least one of an historical approval rate for the respective prescription product by all respective prescribing healthcare professionals and an historical approval rate for the respective prescription product by a respective prescribing healthcare professional.

Clause 9.1 The system of any clause herein, wherein the plurality of prescriber scores, the plurality of prescription product scores, and the member scores are associated with a high volume pharmacy.

Clause 10.1 A method for identifying combinations of prescribing healthcare professionals, members, and products for targeted communication, the method comprising:

receiving a plurality of prescriber scores for respective prescribing healthcare professionals; receiving a plurality of prescription product scores for respective prescription products; receiving a plurality of member scores for receptive members, each respective member being associated with at least one of at least one prescription product associated with a prescription product score of the plurality of prescription product scores and at least one prescribing healthcare professional associated with a prescriber score of the plurality of prescriber scores; receiving targeted communication input indicating a prescriber score weight, a prescription product score weight, and a member score weight; identifying at least one combination of a prescribing healthcare professional, a prescription product, and a member using an artificial intelligence engine configured to use at least one machine learning model configured to use the prescriber score, the prescriber score weight, the prescription production score, the prescription product score weight, the member score, and the member score weight; and generating, for display, an output indicating at least the at least one combination of a prescribing healthcare professional, a prescription product, and a member.

Clause 11.1 The method of any clause herein, wherein each prescribing score of the plurality of prescribing scores is determined based on prescription data associated with a plurality of prescriptions written by a respective prescribing healthcare professional.

Clause 12.1 The method of any clause herein, wherein the prescription data of a respective prescribing healthcare professional includes prescription product data associated with one or more prescriptions of the prescription data, prescriber data associated the respective prescribing healthcare professional, and member identification data associated with the one or more prescriptions of the prescription data.

Clause 13.1 The method of any clause herein, wherein the prescription product data includes at least prescription approval rate data for prescription products of the prescription product data and prescription turnaround time data for prescription products of the prescription product data.

Clause 14.1 The method of any clause herein, wherein each respective prescription product includes at least one of a prescription drug or a prescription accessory.

Clause 15.1 The method of any clause herein, wherein a prescription product score for a respective prescription product is based on an elapsed period between a request for refilling a prescription associated with the respective prescription product and a respective prescribing healthcare professional writing responding to the request.

Clause 16.1 The method of any clause herein, wherein a prescription product score for a respective prescription product is based on a therapeutic index of the respective prescription product.

Clause 17.1 The method of any clause herein, wherein a prescription product score for a respective prescription product is based on at least one of an historical approval rate for the respective prescription product by all respective prescribing healthcare professionals and an historical approval rate for the respective prescription product by a respective prescribing healthcare professional.

Clause 18.1 The method of any clause herein, wherein the plurality of prescriber scores, the plurality of prescription product scores, and the member scores are associated with a high volume pharmacy.

Clause 19.1 An apparatus for identifying combinations of prescribing healthcare professionals, members, and products for targeted communication, the apparatus comprising: one or more processors; and at least one memory including instructions that, when executed by the one or more processors, cause the one or more processors, collectively or respectively, to: determine a plurality of prescriber scores for respective prescribing healthcare professionals; determine a plurality of prescription product scores for respective prescription products; determine a plurality of member scores for receptive members, each respective member being associated with at least one of at least one prescription product associated with a prescription product score of the plurality of prescription product scores and at least one prescribing healthcare professional associated with a prescriber score of the plurality of prescriber scores; receive targeted communication input indicating a prescriber score weight, a prescription product score weight, and a member score weight; identify at least one combination of a prescribing healthcare professional, a prescription product, and a member using an artificial intelligence engine configured to use at least one machine learning model configured to use the prescriber score, the prescriber score weight, the prescription production score, the prescription product score weight, the member score, and the member score weight; and generate, for display, an output indicating at least the at least one combination of a prescribing healthcare professional, a prescription product, and a member.

Clause 20.1 The apparatus of any clause herein, wherein the plurality of prescriber scores, the plurality of prescription product scores, and the member scores are associated with a high volume pharmacy.

Clause 1.2 A system for insurance member communication, the system comprising: a processor; and a memory including instructions that, when executed by the processor, cause the processor to: receive a plurality of member communication campaign input files that define a set of pharmacy product exclusion rules and a set of pharmacy product inclusion rules, wherein the plurality of member communication campaign input files are associated with a member communication campaign; generate one or more campaign tables in a campaign table database based the set of pharmacy product exclusion rules and the set of pharmacy product inclusion rules; retrieve, from an insurance claim database and based on the plurality of member communication campaign input files, insurance claim data; populate records of the one or more campaign tables with the insurance claim data; suppress data in the one or more campaign tables based on a set of suppression rules; and for at least one record of the one or more campaign tables: identify a pharmacy used by an associated member to fill one or more prescriptions; identify one or more alternative pharmacies within a predetermined distance from the identified pharmacy; identify at least one product of the one or more prescriptions; identify one or more alternative products associated with the at least one product of the one or more prescriptions; generate a communication data object that includes (i) at least one alternative pharmacy of the one or more alternative pharmacies that meet at least one alternative pharmacy criteria and (ii) at least one alternative product of the one or more alternative products that meet at least one alternative product criteria; and communicate the communication data object to the associated member.

Clause 2.2 The system of any clause herein, wherein pharmacy products associated with the pharmacy product exclusion rules and pharmacy products associated with the pharmacy products inclusion rules include at least one of a prescription drug or a prescription accessory.

Clause 3.2 The system of any clause herein, wherein the insurance claim data includes member data, and wherein the member data includes at least member identification data, member enrollment status data, prescription product data, pharmacy location data, and pharmacy identification data.

Clause 4.2 The system of any clause herein, wherein the instructions further cause the processor to suppress data in the one or more campaign tables based on the set of suppression rules by removing the suppressed data from the one or more campaign tables and loading the suppressed data in a drop table of the campaign tables database.

Clause 5.2 The system of any clause herein, wherein the set of suppression rules is associated with a functional requirement document associated with the member communication campaign.

Clause 6.2 The system of any clause herein, wherein the instructions further cause the processor to identify the one or more alternative pharmacies within a predetermined distance from the identified pharmacy by communicating an address associated with the identified pharmacy to a geo-location identification system and receiving, from the geo-location identification system, the one or more alternative pharmacies.

Clause 7.2 The system of any clause herein, wherein the at least one alternative pharmacy criteria indicates that the at least one alternative pharmacy is in network.

Clause 8.2 The system of any clause herein, wherein the at least one alternative product criteria indicates that the at least one alternative product is a therapeutic alternative for the at least one product of the one or more prescriptions.

Clause 9.2 The system of any clause herein, wherein the instructions further cause the processor to communicate the communication data object to the associated member by generating an electronic communication and transmitting the electronic communication.

Clause 10.2 A method for insurance member communication, the method comprising: receiving a plurality of member communication campaign input files that define a set of pharmacy product exclusion rules and a set of pharmacy product inclusion rules, wherein the plurality of member communication campaign input files are associated with a member communication campaign; generating one or more campaign tables in a campaign table database based the set of pharmacy product exclusion rules and the set of pharmacy product inclusion rules; retrieving, from an insurance claim database and based on the plurality of member communication campaign input files, insurance claim data; populating records of the one or more campaign tables with the insurance claim data; suppressing data in the one or more campaign tables based on a set of suppression rules; and for at least one record of the one or more campaign tables: identifying a pharmacy used by an associated member to fill one or more prescriptions; identifying one or more alternative pharmacies within a predetermined distance from the identified pharmacy; identifying at least one product of the one or more prescriptions; identifying one or more alternative products associated with the at least one product of the one or more prescriptions; generating a communication data object that includes (i) at least one alternative pharmacy of the one or more alternative pharmacies that meet at least one alternative pharmacy criteria and (ii) at least one alternative product of the one or more alternative products that meet at least one alternative product criteria; and communicating the communication data object to the associated member.

Clause 11.2 The method of any clause herein, wherein pharmacy products associated with the pharmacy product exclusion rules and pharmacy products associated with the pharmacy products inclusion rules include at least one of a prescription drug or a prescription accessory.

Clause 12.2 The method of any clause herein, wherein the insurance claim data includes member data, and wherein the member data includes at least member identification data, member enrollment status data, prescription product data, pharmacy location data, and pharmacy identification data.

Clause 13.2 The method of any clause herein, wherein suppressing data in the one or more campaign tables based on the set of suppression rules includes removing the suppressed data from the one or more campaign tables and loading the suppressed data in a drop table of the campaign tables database.

Clause 14. The method of any clause herein, wherein the set of suppression rules is associated with a functional requirement document associated with the member communication campaign.

Clause 15.2 The method of any clause herein, wherein identifying the one or more alternative pharmacies within a predetermined distance from the identified pharmacy includes communicating an address associated with the identified pharmacy to a geo-location identification system and receiving, from the geo-location identification system, the one or more alternative pharmacies.

Clause 16.2 The method of any clause herein, wherein the at least one alternative pharmacy criteria indicates that the at least one alternative pharmacy is in network.

Clause 17.2 The method of any clause herein, wherein the at least one alternative product criteria indicates that the at least one alternative product is a therapeutic alternative for the at least one product of the one or more prescriptions.

Clause 18.2 The method of any clause herein, wherein communicating the communication data object to the associated member includes generating an electronic communication and transmitting the electronic communication.

Clause 19.2 An apparatus for insurance member communication, the apparatus comprising: one or more processors; and at least one memory including instructions that, when executed by the one or more processors, cause the one or more processors, collectively or respectively, to: generate one or more campaign tables in a campaign table database based a set of pharmacy product exclusion rules and a set of pharmacy product inclusion rules; retrieve, from an insurance claim database, insurance claim data; populate records of the one or more campaign tables with the insurance claim data; suppress data in the one or more campaign tables based on a set of suppression rules by removing the suppressed data from the one or more campaign tables and loading the suppressed data in a drop table of the campaign tables database; and for at least one record of the one or more campaign tables: identify a pharmacy used by an associated member to fill one or more prescriptions; identify one or more alternative pharmacies within a predetermined distance from the identified pharmacy; identify at least one product of the one or more prescriptions; identify one or more alternative products associated with the at least one product of the one or more prescriptions; generate a communication data object that includes (i) at least one alternative pharmacy of the one or more alternative pharmacies that meet at least one alternative pharmacy criteria and (ii) at least one alternative product of the one or more alternative products that meet at least one alternative product criteria; and communicate the communication data object to the associated member.

Clause 20.2 The apparatus of any clause herein, wherein the communication data object is associated with an electronic communication.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Implementations of the systems, algorithms, methods, instructions, etc., described herein may be realized in hardware, software, or any combination thereof. The hardware may include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

What is claimed is:

1. A system for determining a propensity of a prescribing healthcare professional to write a prescription for home delivery, the system comprising:
a processor; and
a memory including instructions that, when executed by the processor, cause the processor to:
receive prescription data associated with a plurality of prescriptions written by a prescribing healthcare professional;
determine a prescriber score for the prescribing healthcare professional based on the prescription data;
determine a prescription product score for each prescription product in each respective prescription of the plurality of prescriptions associated with the prescription data;
retrieve, from a member database, member data associated with respective members of a plurality of members represented in the member database, wherein the respective members have a treatment association with the prescribing healthcare professional;
determine a member score for each respective member of the plurality of members based on at least one home delivery factor;
based on the prescriber score, each prescription product score, and each member score, determine, using an artificial intelligence engine configured to use at least one machine learning model, a prescriber propensity score for the prescribing healthcare professional; and
in response to a determination that the prescriber propensity score is greater than a first threshold:
identify respective members having a member score that is greater than a second threshold; and
generate, for display, an output indicating at least the prescriber propensity score and a list of identified respective members.

2. The system of claim 1, wherein the prescription data includes prescription product data associated with one or more prescriptions of the prescription data, prescriber data associated the prescribing healthcare professional, and member identification data associated with the one or more prescriptions of the prescription data.

3. The system of claim 2, wherein the prescription product data includes at least prescription approval rate data for prescription products of the prescription product data and prescription turnaround time data for prescription products of the prescription product data.

4. The system of claim 1, wherein each prescription product includes at least one of a prescription drug or a prescription accessory.

5. The system of claim 1, wherein the prescription product score for a respective prescription product is based on an elapsed period between a request for refilling a prescription associated with the respective prescription product and the prescribing healthcare professional writing responding to the request.

6. The system of claim 1, wherein the prescription product score for a respective prescription product is based on a therapeutic index of the respective prescription product.

7. The system of claim 1, wherein the prescription product score for a respective prescription product is based on at least one of an historical approval rate for the respective prescription product by all prescribing healthcare professionals and an historical approval rate for the respective prescription product by the prescribing healthcare professional.

8. The system of claim 1, wherein the at least one home delivery factor includes at least one of a digital inclination, a disease state, a market segment, and a geographic location.

9. A method for determining a propensity of a prescribing healthcare professional to write a prescription for home delivery, the method comprising:
receiving prescription data associated with a plurality of prescriptions written by a prescribing healthcare professional;
determining a prescriber score for the prescribing healthcare professional based on the prescription data;
determining a prescription product score for each prescription product in each respective prescription of the plurality of prescriptions associated with the prescription data;
retrieving, from a member database, member data associated with respective members of a plurality of members represented in the member database, wherein the respective members have a treatment association with the prescribing healthcare professional;
determining a member score for each respective member of the plurality of members based on at least one home delivery factor;
based on the prescriber score, each prescription product score, and each member score, determining, using an artificial intelligence engine configured to use at least one machine learning model, a prescriber propensity score for the prescribing healthcare professional; and
in response to a determination that the prescriber propensity score is greater than a first threshold:
identifying respective members having a member score that is greater than a second threshold; and
generating, for display, an output indicating at least the prescriber propensity score and a list of identified respective members.

10. The method of claim 9, wherein the prescription data includes prescription product data associated with one or more prescriptions of the prescription data, prescriber data associated the prescribing healthcare professional, and member identification data associated with the one or more prescriptions of the prescription data.

11. The method of claim 10, wherein the prescription product data includes at least prescription approval rate data for prescription products of the prescription product data and prescription turnaround time data for prescription products of the prescription product data.

12. The method of claim 9, wherein each prescription product includes at least one of a prescription drug or a prescription accessory.

13. The method of claim 9, wherein the prescription product score for a respective prescription product is based on an elapsed period between a request for refilling a prescription associated with the respective prescription product and the prescribing healthcare professional writing responding to the request.

14. The method of claim 9, wherein the prescription product score for a respective prescription product is based on a therapeutic index of the respective prescription product.

15. The method of claim 9, wherein the prescription product score for a respective prescription product is based on at least one of an historical approval rate for the respective prescription product by all prescribing healthcare professionals and an historical approval rate for the respective prescription product by the prescribing healthcare professional.

16. The method of claim 9, wherein the at least one home delivery factor includes at least one of a digital inclination, a disease state, a market segment, and a geographic location.

17. An apparatus for determining a propensity of a prescribing healthcare professional to write a prescription for home delivery, the apparatus comprising:
one or more processors; and
at least one memory including instructions that, when executed by the one or more processors, cause the one or more processors, collectively or respectively, to:
determine a prescriber score for a prescribing healthcare professional based on prescription data associated with a plurality of prescriptions written by the prescribing healthcare professional;
determine a prescription product score for each prescription product in each respective prescription of the plurality of prescriptions associated with the prescription data;
retrieve, from a member database, member data associated with respective members of a plurality of members represented in the member database, wherein the respective members have a treatment association with the prescribing healthcare professional;
determine a member score for each respective member of the plurality of members based on at least one home delivery factor;
based on the prescriber score, each prescription product score, and each member score, determine, using an artificial intelligence engine configured to use at least one machine learning model, a prescriber propensity score for the prescribing healthcare professional; and
in response to a determination that the prescriber propensity score is greater than a first threshold:
identify respective members having a member score that is greater than a second threshold; and
generate, for display, an output indicating at least the prescriber propensity score and a list of identified respective members.

18. The apparatus of claim 17, wherein the prescription data includes prescription product data associated with one or more prescriptions of the prescription data, prescriber data associated the prescribing healthcare professional, and member identification data associated with the one or more prescriptions of the prescription data.

19. The apparatus of claim 18, wherein the prescription product data includes at least prescription approval rate data for prescription products of the prescription product data and prescription turnaround time data for prescription products of the prescription product data.

20. The apparatus of claim 17, wherein the prescription data is associated with a high volume pharmacy.
